(19) United States
(12) Patent Application Publication
(10) Pub. No.: US 2022/0273587 A1
(43) Pub. Date: Sep. 1, 2022

(54) ALKALIZATION OF URINARY BLADDER WALL PRIOR TO TREATMENT WITH INTRAVESICAL HEPARIN AND ALKALINIZED LIDOCAINE TO ENHANCE RELIEF OF BLADDER PAIN SYMPTOMS

(71) Applicant: C. Lowell PARSONS, Henderson, NV (US)

(72) Inventor: C. Lowell PARSONS, Henderson, NV (US)

(21) Appl. No.: 17/627,044

(22) PCT Filed: Jul. 14, 2020
(86) PCT No.: PCT/US2020/041977

§ 371 Date: Jan. 13, 2022

Related U.S. Application Data
(60) Provisional application No. 62/875,745, filed on Jul. 18, 2019.

(51) Int. Cl. A61K 31/137; A61K 45/06

(52) U.S. Cl. CPC A61K 31/137; A61K 45/06; A61M 19/00

(57) ABSTRACT

The present invention is directed to an improved method of treating diseases and conditions associated with bladder pain, particularly interstitial cystitis. In general, a method according to the present invention comprises: (1) instilling into the bladder of a subject with a disease or condition associated with bladder pain a quantity and concentration of a physiologically compatible buffer to raise the pH of the bladder epithelium sufficiently to reduce acidosis associated with the disease or condition; (2) allowing the physiologically compatible buffer of (a) to remain in the bladder for a period sufficiently long to enable the buffer to raise the pH; (3) removing the physiologically compatible buffer of (1) from the bladder; and (4) instilling into the bladder a quantity of a composition comprising: (i) a heparinoid; (ii) a local anesthetic; and (iii) a buffer, wherein the composition has a pH of from about 7.0 to about 7.4 to treat the disease or condition associated with bladder pain. The present invention also is directed to kits for practicing the method.

### CROSS-REFERENCE TO RELATED APPLICATION

This application claims the benefit of U.S. Provisional Patent Application Ser. No. 62/875,745, entitled “Alkalization of Urinary Bladder Wall Prior to Treatment with Intravesical Heparin and Alkalinized Lidocaine to Enhance Relief of Bladder Pain Symptoms,” by Dr. C. Lowell Parsons, filed Jul. 18, 2019, the contents of which are incorporated herein by reference in their entirety.

### FIELD OF THE INVENTION

This invention is directed to compositions and methods for alkalization of the urinary bladder wall prior to treatment with agents such as, but not limited to, heparinoids such as heparin and an alkalinized local anesthetic such as lidocaine, for the treatment of bladder pain symptoms.

### BACKGROUND OF THE INVENTION

Interstitial cystitis (IC), also frequently known as bladder pain syndrome or hypersensitive bladder syndrome, is a chronic progressive disorder of the lower urinary tract that causes urinary urgency and frequency and/or pelvic pain. American Urology Association defines IC/BPS as “an unpleasant sensation (pain, pressure, discomfort) perceived to be related to the urinary bladder, associated with lower urinary tract symptoms of more than six weeks duration, in the absence of infection or other identifiable causes.” For many years, urologists regarded IC/BPS as a rare disease for which they had no broadly effective treatment. In fact, the condition is quite common. In 1999, prevalence in the United States was estimated at 750,000 cases (Curhan, et al. J Urol 161(2):549-552 (1999)). However current estimates from the RAND Interstitial Cystitis Epidemiology (RICE) study suggests the true prevalence of IC/BPS is estimated to be 2.7% to 6.53% (approximately 3.3 to 7.9 million US women age 18 or older) and 2.9% to 4.2% (approximately 2.0 to 4.6 million US men age 18 or older) (Berry S H et al. J Urol 2011; 186: 540; and Suskind A M et al. J Urol 2013; 189: 141). In addition overactive bladder, urethral syndrome, prostatitis, and gynecologic chronic pelvic pain syndrome affect millions of patients that also result in bladder symptoms of urgency, frequency, incontinence and or pelvic pain with no effective therapy and all these syndromes share similar symptoms and likely a common pathophysiology with traditionally diagnosed IC (Parsons, C L Int Br J Urol December, 2010); there is a need for more broadly effective treatments for these conditions, particularly when these conditions are severe.

Millions of women and many men suffer from bladder symptoms of urgency, frequency of urination, incontinence and bladder generated pelvic pain. This includes people with overactive bladder, prostatitis, urethritis, urethral syndrome, radiation cystitis and interstitial cystitis (bladder pain syndrome). It has been demonstrated that a combination of a heparinoid, such as, but not limited to, heparin and sodium pentosanpolysulfate, plus an alkalinized local anesthetic, such as, but not limited to, lidocaine, administered intravesically will relieve these symptoms in interstitial cystitis patients (C. L Parsons, “Successful Downregulation of Bladder Sensory Nerves with Combination of Heparin and Alkalinized Lidocaine in Patients with Interstitial Cystitis,” Urology 65: 45-48 (2005)). The anesthetic lidocaine in its free base (unprotonated) form is insoluble in water and hence is protonated with hydrochloric acid, to form lidocaine hydrochloride which is readily soluble in water or aqueous solutions. However, the ionic form of lidocaine does not penetrate the lipid bilayers of cell membranes efficiently, such as the bladder epithelium and the membranes of nerve cells, because charged molecules such as the protonated form of lidocaine do not penetrate efficiently through the highly hydrophobic lipid bilayers of cell membranes.

Accordingly, the lidocaine is alkalized and combined with a heparinoid such as, but not limited to, heparin or sodium pentosanpolysulfate, to raise the pH above 7 (the lidocaine is soluble and stable in this composition if mixed properly) and by doing so, the lidocaine absorption into the bladder wall increases approximately twofold (C. L. Parsons et al., “Heparin and Alkalized Lidocaine Versus Alkalized Lidocaine for Treatment of Interstitial Cystitis,” Canadian J. Urol. 22: 7739-7743 (2015)) and results in better symptom relief in the patient. About 50-55% of patients have a good response to this solution; however, about 35-40% have minimal or no response to the heparin/lidocaine treatment (C. L. Parsons et al., “Alkalinized Lidocaine and Heparin Provide Immediate Relief of Pain and Urgency in Patients with Interstitial Cystitis,” J. Sex. Med. 9: 207-212 (2012)). The reasons why this fraction of patients does not respond are unknown, but if these reasons were discovered, then it could lead to substantial improvement in the efficacy of the heparin/lidocaine mixture and thus substantial improvement in the treatment of interstitial cystitis and other diseases and conditions associated with bladder pain as stated above.

The reason that interstitial cystitis patients develop bladder symptoms is because their normally impermeable bladder epithelium is impaired, and this allows potassium, which is very concentrated in urine, to “leak” into the bladder wall, depolarizing muscles and nerves as well as causing tissue damage and inflammation (C. L. Parsons, “The Role of a Leaky Epithelium and Potassium in the Generation of Bladder Symptoms in Interstitial Cystitis/Overactive Bladder, Urethral Syndrome, Prostatitis and Gynaecological Chronic Pelvic Pain,” BJU Int. 107: 370-375 (2011)). To investigate the non-responsiveness of many interstitial cystitis patients to heparin/alkalinized lidocaine, it was hypothesized that perhaps the inflammation occurring in the bladder wall caused by the potassium, especially in the patients with more severe disease, results in a drop of the normal tissue pH, which is about 7.3 to 7.4, to a value of perhaps 7.0 or lower.

There has been a previous report of treating interstitial cystitis patients (R. Henry et al., “Absorption of Alkalized Intravesical Lidocaine in Normal and Inflamed Bladders: A Simple Method for Improving Bladder Anesthesia,” J. Urol. 165: 1900-1903 (2001)) with lidocaine and sodium bicarbonate. This method involved placing first 10 mL of lidocaine (5 mg/kg in 5% dextrose/water, about 220 to 240 μg) into the urinary bladder of interstitial cystitis patients and then immediately adding 10 mL of 8.4% sodium bicarbonate. Since adding sodium bicarbonate to lidocaine is known to precipitate the lidocaine, the bicarbonate was given sequentially to prevent the lidocaine from precipitating in the syringe or the catheter before it reached the bladder lumen. The bottom line is that the lidocaine and bicarbonate were given as a combination to the bladder with the belief that the alkalinized lidocaine would better absorb into the bladder wall to provide symptom relief and hence only one treatment was performed.

Therefore, there is a need for improved therapeutic methods to treat interstitial cystitis and other diseases and conditions associated with bladder pain, particularly improved therapeutic methods that can provide relief to that substantial proportion of patients whose symptoms are not relieved by currently available methods.

### SUMMARY OF THE INVENTION

The methods of the present invention, as described in detail below, meet the need for providing improved therapeutic methods to treat interstitial cystitis and other diseases and conditions associated with bladder pain, particularly improved therapeutic methods that can provide relief to that substantial proportion of patients whose symptoms are not relieved by currently available methods. In particular, the methods introduce a step of buffering the bladder to relieve acidosis that is associated with inflammation.

One aspect of the present invention is a method for treating a disease or condition associated with bladder pain comprising the steps of:

(1) instilling into the bladder of a subject with a disease or condition associated with bladder pain a quantity and concentration of a physiologically compatible buffer to raise the pH of the bladder epithelium and the interstitium of the bladder wall sufficiently to reduce acidosis associated with the disease or condition;

(2) allowing the physiologically compatible buffer of (1) to remain in the bladder for a period sufficiently long to enable the buffer to raise the bladder tissue pH;

(3) removing the physiologically compatible buffer of (2) from the bladder to prevent the precipitation of subsequently added local anesthetic; and

(4) instilling into the bladder a quantity of a composition comprising: (i) a heparinoid; (ii) a local anesthetic; and (iii) a buffer, wherein the composition has a pH of from about 7.0 to about 7.4 to treat the disease or condition associated with bladder pain.

Typically, the disease or condition associated with bladder pain and treatable by the method is selected from the group consisting of bacterial cystitis, fungal/yeast cystitis, vulvar vestibulitis, vulvodynia, dyspareunia, urethral syndrome, and endometriosis in women; prostatitis and chronic pelvic pain syndrome in men; and radiation-induced cystitis, chemotherapy-induced cystitis, interstitial cystitis, and overactive bladder in men or women. In particular, the disease or condition associated with bladder pain and treatable by the method is interstitial cystitis.

Typically, the physiologically compatible buffer of step (1) is selected from the group consisting of phosphate buffer, bicarbonate buffer, Tris (Tris(hydroxymethyl)aminomethane) buffer, MOPS buffer (3-(N-morpholino)propanesulfonic acid), HEPES (N-(2-hydroxyethyl)piperazine-N-(2-ethanesulfonic acid) buffer, ACES (2-[(2-amino-2-oxoethyl)amino]ethanesulfonic acid) buffer, ADA (N-(2-acetamido)2-iminodiacetic acid) buffer, AMPSO (3-[(1,1-dimethyl-2-hydroxyethyl)amino]-2-propanesulfonic acid) buffer, BES (N,N-bis(2-hydroxyethyl)-2-aminoethanesulfonic acid buffer, Bicine (N,N-bis(2-hydroxyethylglycine) buffer, Bis-Tris (bis-(2-hydroxyethyl)imino-tris(hydroxymethyl)methane buffer, CAPS (3-(cyclohexylamino)-1-propanesulfonic acid) buffer, CAPSO (3-(cyclohexylamino)-2-hydroxy-1-propanesulfonic acid) buffer, CHES (2-(N-cyclohexylamino)ethanesulfonic acid) buffer, DIPSO (3-[N,N-bis(2-hydroxyethyl)amino]-2-hydroxy-propanesulfonic acid) buffer, HEPPS (N-(2-hydroxyethylpiperazine)-N′-(3-propanesulfonic acid) buffer, HEPPSO (N-(2-hydroxyethyl)piperazine-N′-(2-hydroxypropanesulfonic acid) buffer, MES (2-(N-morpholino)ethanesulfonic acid) buffer, triethanolamine buffer, imidazole buffer, glycine buffer, ethanolamine buffer, MOPSO (3-(N-morpholino)-2-hydroxypropanesulfonic acid) buffer, PIPES (piperazine-N,N′-bis(2-ethanesulfonic acid) buffer, POPSO (piperazine-N,N′-bis(2-hydroxypropaneulfonic acid) buffer, TAPS (N-tris[hydroxymethyl)methyl-3-aminopropanesulfonic acid) buffer; TAPSO (3-[N-tris(hydroxymethyl)methylamino]-2-hydroxy-propanesulfonic acid) buffer, TES (N-tris(hydroxymethyl)methyl-2-aminoethanesulfonic acid) buffer, tricine (N-tris(hydroxymethyl)methylglycine buffer), 2-amino-2-methyl-1,3-propanediol buffer, and 2-amino-2-methyl-1-propanol buffer, and a combination thereof. Preferably, the physiologically compatible buffer is selected from the group consisting of phosphate buffer, Tris buffer, and bicarbonate buffer. When the buffer is bicarbonate buffer, the buffer is typically sodium bicarbonate.

Typically, the volume of the phosphate buffer, Tris buffer, or sodium bicarbonate buffer is from about 40 mL to about 50 mL. Preferably, the volume of the phosphate buffer, Tris buffer, or sodium bicarbonate buffer is from about 47.5 mL to about 52.5 mL. More preferably, the volume of the phosphate buffer, Tris buffer, or sodium bicarbonate buffer is about 50 mL. However, in case of patient intolerance to this higher volume a lower volume is added to the bladder to the maximum tolerated.

Typically, the concentration of the phosphate buffer, Tris buffer, or sodium bicarbonate buffer is from about 0.90 M to about 1.10 M. Preferably, the concentration of the phosphate buffer, Tris buffer, or sodium bicarbonate buffer is from about 0.95 M to about 1.05 M. More preferably, the concentration of the phosphate buffer, Tris buffer, or sodium bicarbonate buffer is about 1.00 M.

Generally, the sufficiently long period of time to enable the buffer to raise the pH in the bladder wall is about 10 to 15 minutes or more as tolerated by the patient. Preferably, the sufficiently long period of time to enable the buffer to raise the pH in the bladder is about 15 minutes to about 20 minutes.

Typically, the composition administered in step (4) includes the heparinoid in a quantity sufficient to treat the urinary tract disease or condition associated with bladder pain. Typically, the composition administered in step (4) includes the local anesthetic in a quantity sufficient to treat the urinary tract disease or condition associated with bladder pain. Typically, the composition administered in step (4) includes the buffer in a quantity such that from about 2% to about 45% of the local anesthetic is present in the composition in the free base (uncharged) form rather than the protonated (charged) form.

Typically, the heparinoid of the composition administered in step (4) is selected from the group consisting of heparin, chondroitin sulfate, heparan sulfate, hyaluronic acid, keratan sulfate, dermatan sulfate, hyaluronan, sodium pentosanpolysulfate, dalteparin and enoxaparin. Preferably, the heparinoid is selected from the group consisting of heparin, heparan sulfate, chondroitin sulfate, hyaluronic acid, and sodium pentosanpolysulfate. When the heparinoid is heparin, it is preferably heparin sodium. Typically, the quantity of heparin in the composition administered in step (4) is from about 1000 units to about 250,000 units per unit dose of the composition. In particular alternatives, the quantity of heparin in the composition administered in step (4) is about 40,000 units, about 50,000 units, or about 60,000 units of heparin per unit dose of the composition.

Typically, the local anesthetic of the composition administered in step (4) is a local anesthetic of the amide class that possesses a protonatable tertiary amino group that can form a positively charged quaternary amino group when protonated. Preferably, the local anesthetic is selected from the group consisting of lidocaine, bupivacaine, etidocaine, mepivacaine, ropivacaine, dibucaine, dexivacaine, levobupivacaine, pyrrocaine, trimecaine, and rodocaine. More preferably, the local anesthetic is selected from the group consisting of lidocaine, bupivacaine, and mepivacaine. A particularly preferred local anesthetic is lidocaine, in particular lidocaine hydrochloride. When the local anesthetic is lidocaine, typically the quantity of lidocaine in the composition of step (d) is from about 10 mg to about 400 mg per unit dose of the composition. Suitable compositions include compositions including 10 mL of 1% lidocaine per unit dose of the composition or 16 mL of 2% lidocaine per unit dose of the composition. In another alternative, suitable compositions can include compositions including 200 mg of lidocaine hydrochloride per unit dose of the composition.

Typically, the buffer of the composition administered in step (4) is selected from the group consisting of phosphate buffer, bicarbonate buffer, Tris (Tris(hydroxymethyl)aminomethane) buffer, MOPS buffer (3-(N-morpholino)propanesulfonic acid), HEPES (N-(2-hydroxyethyl)piperazine-N-(2-ethanesulfonic acid) buffer, ACES (2-[(2-amino-2-oxoethyl)amino]ethanesulfonic acid) buffer, ADA (N-(2-acetamido)2-iminodiacetic acid) buffer, AMPSO (3-[(1,1-dimethyl-2-hydroxyethyl)amino]-2-propanesulfonic acid) buffer, BES (N,N-bis(2-hydroxyethyl)-2-aminoethanesulfonic acid buffer, Bicine (N,N-bis(2-hydroxyethylglycine) buffer, Bis-Tris (bis-(2-hydroxyethyl)imino-tris(hydroxymethyl)methane buffer, CAPS (3-(cyclohexylamino)-1-propanesulfonic acid) buffer, CAPSO (3-(cyclohexylamino)-2-hydroxy-1-propanesulfonic acid) buffer, CHES (2-(N-cyclohexylamino)ethanesulfonic acid) buffer, DIPSO (3-[N,N-bis(2-hydroxyethyl)amino]-2-hydroxy-propanesulfonic acid) buffer, HEPPS (N-(2-hydroxyethylpiperazine)-N′-(3-propanesulfonic acid) buffer, HEPPSO (N-(2-hydroxyethyl)piperazine-N′-(2-hydroxypropanesulfonic acid) buffer, MES (2-(N-morpholino)ethanesulfonic acid) buffer, triethanolamine buffer, imidazole buffer, glycine buffer, ethanolamine buffer, MOPSO (3-(N-morpholino)-2-hydroxypropanesulfonic acid) buffer, PIPES (piperazine-N,N′-bis(2-ethanesulfonic acid) buffer, POPSO (piperazine-N,N′-bis(2-hydroxypropaneulfonic acid) buffer, TAPS (N-tris[hydroxymethyl)methyl-3-aminopropanesulfonic acid) buffer; TAPSO (3-[N-tris(hydroxymethyl)methylamino]-2-hydroxy-propanesulfonic acid) buffer, TES (N-tris(hydroxymethyl)methyl-2-aminoethanesulfonic acid) buffer, tricine (N-tris(hydroxymethyl)methylglycine buffer), 2-amino-2-methyl-1,3-propanediol buffer, and 2-amino-2-methyl-1-propanol buffer, or a combination thereof. Preferably, the buffer is selected from the group consisting of bicarbonate buffer, phosphate buffer, and Tris buffer. When the buffer is bicarbonate buffer, typically the bicarbonate buffer is sodium bicarbonate.

A particularly preferred composition administered in step (4) of the method is alkalinized lidocaine/heparin in phosphate buffer that contains 240 mg of lidocaine and 60,000 units of heparin in phosphate buffer with a pH of 7.2-7.3. Preferably, this composition is administered in about 20 mL per unit dose.

In some alternatives, the composition administered in step (4) of the method includes at least one additional component. The at least one additional component can be: (i) an osmolar component that provides an isotonic or nearly isotonic solution compatible with human cells and blood; (ii) an antibacterial agent; (iii) an antifungal agent; (iv) a vasoconstrictor; (v) a compound that enables persistence of the composition to the surface of the bladder epithelium; (vi) a preservative; or (vii) an anti-inflammatory agent.

Typically, the volume of the composition administered in step (4) of the method is from about 15 mL to about 25 mL. Preferably, the volume of the composition administered in step (4) of the method is from about 17.5 mL to about 22.5 mL. More preferably, the volume of the composition administered in step (4) of the method is about 20 mL.

Typically, the composition administered in step (4) of the method is left in the bladder for from about 40 minutes to about 50 minutes. Preferably, the composition administered in step (4) of the method is left in the bladder for from about 42.5 minutes to about 47.5 minutes. More preferably, the administered in step (4) of the method is left in the bladder for about 45 minutes.

In one alternative, the method further comprises the step of administering a therapeutically effective quantity of an additional agent to control the symptoms of interstitial cystitis or other diseases or conditions associated with bladder pain. The additional agent can be selected from the group consisting of an oral non-steroidal anti-inflammatory drug, detroloxybutynin chloride, tolterodine, mesna, and dimethylsulfoxide.

Another aspect of the present invention is a multipart kit for the treatment of a disease or condition associated with bladder pain comprising:

(1) one or more unit doses, separately packaged, of a physiologically compatible buffer to raise the pH of the bladder epithelium and bladder interstitial tissue sufficiently to reduce acidosis associated with the disease and condition;

(2) one or more unit doses, separately packaged, of a composition comprising: (i) a heparinoid; (ii) a local anesthetic; and (iii) a buffer, wherein the composition has a pH of from about 7.0 to about 7.4 to treat the disease or condition associated with bladder pain; and

(3) instructions for use of the kit.

Suitable alternatives for the physiologically compatible buffer of (1) and the compositions of (2) are as described above. The volumes of the physiologically compatible buffer of (1) and the composition of (2), per unit dose, are also as described above.

### DETAILED DESCRIPTION OF THE INVENTION

The present invention provides improved therapeutic methods for treatment of interstitial cystitis and other diseases and conditions associated with bladder pain, particularly by improving the bioavailability of local anesthetics such as lidocaine administered in a heparin/alkalinized lidocaine composition. These improved therapeutic methods are particularly useful for patients with severe interstitial cystitis.

According to the present invention, pretreating the urinary bladder with a large volume and concentration of an alkalized buffer allows the buffering material time to diffuse into the bladder wall (readily accomplished because the urothelium of interstitial cystitis is defective and allows small solutes to leak through the urothelium), thus raising the pH of the bladder interstitium that is likely acidotic from the ongoing inflammation that is present in the tissue. The buffer then raises the pH of the bladder wall back toward the normal physiological pH of about 7.4, a perfect environment for the lidocaine free base, unprotonated, form to exist which results in more rapid (and higher) absorption into the bladder nerves. This results in a significantly greater and longer anesthetic effect that helps to downregulate the nerves.

In general, the occurrence of inflammation is known to result in acid production and therefore causes lower pH levels, sometimes even below 7.0. In systemic infections that are severe, the pH level in blood may drop from 7.4 to 7.0 or lower, a condition known as acidosis, which can be life-threatening, requiring immediate treatment to restore a normal pH value. In local infections such abscesses the surrounding tissue has a drop in pH; local anesthetics have little or no effect in such acidic environments due to the protonation of the local anesthetic that produces a charged form of the local anesthetic that cannot readily pass through the lipid bilayers of the membranes of the affected cells, and thus the abscess often has to be lanced without anesthesia. There is no practical way to measure the pH in the bladder wall in patients with interstitial cystitis or other diseases or conditions associated with bladder pain, so that it is unknown if the tissue pH is abnormally low. However, if the patients, particularly the patients with severe interstitial cystitis, do have, in effect, a local acidosis in the bladder wall, then one would expect lidocaine, or another local anesthetic that can exist in protonated and unprotonated forms, to have minimal or no effect. This might explain why the more severe patients do not respond well or even do not respond at all to intravesical lidocaine. As a corollary, one would not expect the cocktails used in the bladder that contain alkalinized lidocaine and heparin to have sufficient alkalinizing buffer to affect the interstitial tissues of the urinary bladder for the obvious reason that such cocktails simply do not work in severe patients.

To test the hypothesis that the patients with severe disease have local inflammation serious enough to lower tissue pH, a new protocol was developed to treat interstitial cystitis patients. The main purpose was to attempt to alkalinize the bladder interstitium by pretreating the bladder with a large volume and a high concentration of an alkalinizing buffer (8.4% sodium bicarbonate was used). To accomplish this, 50 mL of 8.4% sodium bicarbonate was placed into the bladder of the interstitial cystitis patient and left for a period of time to allow the low molecular weight solutes to absorb into the bladder wall. This solution was left indwelling for 15 to 20 minutes depending on patient tolerability. After the allotted time, the sodium bicarbonate was drained with a catheter to prevent the subsequently added anesthetic from being precipitated by this large volume of buffer and 20 mL of alkalinized lidocaine/heparin in phosphate buffer that contained 240 mg of lidocaine and 60,000 units of heparin in phosphate buffer with a pH of 7.2-7.3 was instilled and left for 45 minutes and the patient responses were recorded.

The results were as follows. Fifteen interstitial cystitis patients who had previously received the heparin/alkalinized lidocaine solution as described above on multiple occasions but who had never responded to the treatment entered and completed the trial. Fourteen out of the fifteen responded dramatically (the patient reported at least a drop of 50% in their overall symptoms, an outcome measure widely used in interstitial cystitis studies (C. L. Parsons et al., “Heparin and Alkalized Lidocaine Versus Alkalized Lidocaine for Treatment of Interstitial Cystitis,” Canadian J. Urol. 22: 7739-7743 (2015)). The average pain drop on an analog scale was in excess of 50%. This is an amazing response rate in subjects who had previously never responded to therapy and the length of the response was an average of 5-12 hours, which is also a more dramatic length of response than was expected. Interestingly, all but one patient reported substantial burning while being pretreated with the sodium bicarbonate; however, this resolved within minutes after the second treatment with the heparin/alkalinized solution. This burning response likely occurred because the bicarbonate was being absorbed, and, not surprisingly, a sudden change in tissue pH might readily depolarize the pain fibers (salt in the wound). Also of interest, the only patient who did not experience any burning or unpleasant feeling with the initial bicarbonate treatment had no response to the second treatment and this suggested that she did not have pelvic pain of bladder origin. These observations support the concept that there is an abnormally low pH in the bladder wall in interstitial cystitis patients. The highly significant anesthetic effect also strongly supports this hypothesis, since the lidocaine is more readily absorbed into the bladder pain fibers if the local pH in the tissue is alkaline; as stated above, lidocaine at an alkaline pH is substantially uncharged and more readily crosses the lipid bilayer of the cell membrane, while lidocaine at an acidic pH is positively charged because of the protonation of an amino moiety to form a quaternary ammonium moiety. In addition, the better nerve absorption of lidocaine should and did result in a much better and longer duration of pain relief.

Accordingly, the present invention is directed to the treatment of bladder symptoms in interstitial cystitis patients, as well as other patients experiencing bladder pain as detailed below, with two separate intravesical solutions. The first stage, employing a first intravesical solution, uses a volume of alkalizing buffer sufficient to alkalinize the bladder interstitium. The second stage, employing a second intravesical solution, uses an alkalinized solution of a heparinoid and a local anesthetic, which is instilled into the buffer to anesthetize the bladder nerves to relieve symptoms.

For the first stage, the interstitial tissue of the bladder in interstitial cystitis patents may well be acidotic due to the chronic and severe inflammation present in most of these patients. In an acidotic environment, lidocaine will not absorb well or at all into sensory nerve fibers associated with pain and urgency located in the bladder wall and hence no or minimal symptom relief will be experienced in such patients. The first stage, therefore, is performed with a significant amount of alkalinizing buffer over a period of time sufficient to allow it to diffuse into the bladder wall and alkalize the tissue. This will keep the alkalinized lidocaine or other local anesthetic, as described below, in the second solution in its free base (unprotonated) form so that it will readily diffuse into the sensory nerve endings and relieve symptoms.

For the second stage, after the buffering agent in the first stage is removed in order to prevent this high volume of buffer from precipitating the subsequently added local anesthetic, a second solution containing alkalinized lidocaine, or, as detailed below, another alkalinized local anesthetic, and heparin, or as detailed below, another heparinoid, is placed into the bladder, typically by instillation through a catheter. The alkalization of the lidocaine or other local anesthetic allows the local anesthetic to go into its hydrophobic free base form and increases its absorption through the lipid membranes of the bladder epithelium. The key to the present invention, which has not been known or described previously, is that the pretreatment of the bladder wall with an alkalinizing buffer, such as, but not necessarily limited to, sodium bicarbonate, can correct bladder wall acidosis in patients with interstitial cystitis or other conditions resulting in bladder pain. This will allow the lidocaine or other local anesthetic to stay in its free base form, including in any urine present, to promote absorption into the bladder wall so that it can readily diffuse into the sensory nerve endings and anesthetize them.

The invention according to the present application, in contrast to alternatives for treatment that have been described previously, is two separate treatments designed to significantly improve bladder symptom response by first alkalinizing the bladder wall and then administering a separate solution to alleviate the symptoms associated with diseases and conditions such as interstitial cystitis. No reports are known that disclose the use of two separate intravesical treatments to alleviate the symptoms of interstitial cystitis patients that provides an alkaline environment for the intralumenal bladder solution and continues this alkaline milieu into the bladder wall tissue.

Bladder pretreatment requires an alkalinizing buffer that could be provided by any standard buffering agent such as sodium bicarbonate, Tris buffer, or phosphate buffer; other alternative buffers that are suitable for use in a physiological context are described below. It must be given as a separate treatment in volumes sufficiently large and concentrations sufficiently high to allow the buffering agent or agents to diffuse into the bladder wall with a dwell time sufficient to accomplish this. Optimally, a volume of 50 mL indwelling in the bladder for 15 or more minutes can be used, as demonstrated by the data described above. The first intravesical solution must be removed before the introduction of the second intravesical solution because the higher concentration and volume of the alkalinizing buffer will precipitate the lidocaine or other local anesthetic, destroying the desired anesthetic effect needed to alleviate the patient's symptoms. For these reasons, one solution could not accomplish the desired effect of alkalinizing the bladder interstitium and simultaneously maintaining an intravesical pH in which the lidocaine, or another alternative local anesthetic as described below, is soluble and stable. In the past, volumes of up to 40 mL of a heparin/lidocaine solution have been used, but the patients still did not respond. As used previously, the amount of buffer in the solution was much lower and obviously not sufficient to alter the acidosis in the bladder wall, particularly for patients with severe interstitial cystitis. As mentioned previously, increasing the volume or concentration of the alkalizing buffer precipitates the lidocaine.

Accordingly, one aspect of the present invention is a method for treating a disease or condition associated with bladder pain comprising the steps of:

(1) instilling into the bladder of a subject with a disease or condition associated with bladder pain a quantity and concentration of a physiologically compatible buffer to raise the pH of the bladder epithelium sufficiently to reduce acidosis associated with the disease and condition;

(2) allowing the physiologically compatible buffer of (1) to remain in the bladder for a period sufficiently long to enable the buffer to raise the pH;

(3) removing the physiologically compatible buffer of (1) from the bladder; and

(4) instilling into the bladder a quantity of a composition comprising: (i) a heparinoid; (ii) a local anesthetic; and (iii) a buffer, wherein the composition has a pH of from about 7.0 to about 7.4 to treat the disease or condition associated with bladder pain.

In a method according to the present invention, the disease or condition associated with bladder pain can be selected from the group consisting of bacterial cystitis, fungal/yeast cystitis, vulvar vestibulitis, vulvodynia, dyspareunia, urethral syndrome, and endometriosis in women; prostatitis and chronic pelvic pain syndrome in men; and radiation-induced cystitis, chemotherapy-induced cystitis, interstitial cystitis, and overactive bladder in men or women. Typically, the disease or condition associated with bladder pain is interstitial cystitis (also known as bladder pain syndrome or hypersensitive bladder syndrome).

In a method according to the present invention, the physiologically compatible buffer of step (1) is typically selected from the group consisting of phosphate buffer, bicarbonate buffer, Tris (Tris(hydroxymethyl)aminomethane) buffer, MOPS buffer (3-(N-morpholino)propanesulfonic acid), HEPES (N-(2-hydroxyethyl)piperazine-N-(2-ethanesulfonic acid) buffer, ACES (2-[(2-amino-2-oxoethyl)amino]ethanesulfonic acid) buffer, ADA (N-(2-acetamido)2-iminodiacetic acid) buffer, AMPSO (3-[(1,1-dimethyl-2-hydroxyethyl)amino]-2-propanesulfonic acid) buffer, BES (N,N-bis(2-hydroxyethyl)-2-aminoethanesulfonic acid buffer, Bicine (N,N-bis(2-hydroxyethylglycine) buffer, Bis-Tris (bis-(2-hydroxyethyl)imino-tris(hydroxymethyl)methane buffer, CAPS (3-(cyclohexylamino)-1-propanesulfonic acid) buffer, CAPSO (3-(cyclohexylamino)-2-hydroxy-1-propanesulfonic acid) buffer, CHES (2-(N-cyclohexylamino)ethanesulfonic acid) buffer, DIPSO (3-[N,N-bis(2-hydroxyethyl)amino]-2-hydroxy-propanesulfonic acid) buffer, HEPPS (N-(2-hydroxyethylpiperazine)-N′-(3-propanesulfonic acid) buffer, HEPPSO (N-(2-hydroxyethyl)piperazine-N′-(2-hydroxypropanesulfonic acid) buffer, MES (2-(N-morpholino)ethanesulfonic acid) buffer, triethanolamine buffer, imidazole buffer, glycine buffer, ethanolamine buffer, MOPSO (3-(N-morpholino)-2-hydroxypropanesulfonic acid) buffer, PIPES (piperazine-N,N′-bis(2-ethanesulfonic acid) buffer, POPSO (piperazine-N,N′-bis(2-hydroxypropaneulfonic acid) buffer, TAPS (N-tris[hydroxymethyl)methyl-3-aminopropanesulfonic acid) buffer; TAPSO (3-[N-tris(hydroxymethyl)methylamino]-2-hydroxy-propanesulfonic acid) buffer, TES (N-tris(hydroxymethyl)methyl-2-aminoethanesulfonic acid) buffer, tricine (N-tris(hydroxymethyl)methylglycine buffer), 2-amino-2-methyl-1,3-propanediol buffer, and 2-amino-2-methyl-1-propanol buffer, and a combination thereof. Typically, the physiologically compatible buffer is selected from the group consisting of phosphate buffer, Tris buffer, and bicarbonate buffer. When the buffer is bicarbonate buffer, typically the counterion of the bicarbonate buffer is sodium. Other counterions can be used in place of sodium, but, because potassium ions can exacerbate the symptoms of interstitial cystitis and other diseases or conditions associated with bladder pain syndrome, potassium counterions should generally be avoided unless it is certain that the disease or condition associated with bladder pain syndrome being treated is one that the presence of potassium ions does not exacerbate.

When the physiologically compatible buffer in step (1) is sodium bicarbonate buffer, typically the volume of sodium bicarbonate buffer is from about 45 mL to about 55 mL. Preferably, the volume of sodium bicarbonate buffer is from about 47.5 mL to about 52.5 mL. More preferably, the volume of sodium bicarbonate buffer is about 50 mL.

When the physiologically compatible buffer in step (1) is sodium bicarbonate buffer, typically, the concentration of sodium bicarbonate buffer is from about 0.90 M to about 1.10 M. Preferably, the concentration of sodium bicarbonate buffer is from about 0.95 M to about 1.05 M. More preferably, the concentration of sodium bicarbonate buffer is about 1.0 M (8.4%).

A particularly preferred combination of concentration and volume of physiologically compatible buffer in step (1), when the buffer is sodium bicarbonate buffer, is about 50 mL of about 8.4% sodium bicarbonate buffer (1.0 M sodium bicarbonate buffer). However, other optimum concentrations and volumes of sodium bicarbonate buffer can be determined for particular patients by a skilled practitioner, dependent on factors such as the size of the bladder, the particular disease or condition being treated, the severity of the disease or condition being treated, the tolerance of the patient to the instillation of large volumes of buffer into the bladder, other diseases or conditions affecting the urinary tract, including the bladder and ureters, and other medications being administered to the patient.

When the physiologically acceptable buffer is a buffer other than sodium bicarbonate buffer, suitable volumes and concentrations can be determined by a skilled practitioner. However, when the buffer is Tris buffer, typically, the concentration of the buffer is from about 0.90 M to about 1.10 M; preferably, the concentration of the buffer is from about 0.95 M to about 1.10 M; and, more preferably, the concentration of the buffer is about 1.0 M; a typical volume of the buffer is from about 45 mL to about 55 mL; a preferable volume of the buffer is from about 47.5 mL to about 52.5 mL; and a more preferable volume of the buffer is about 50 mL. When the buffer is phosphate buffer, typically, the concentration of the buffer is from about 0.90 M to about 1.10 M; preferably, the concentration of the buffer is from about 0.95 M to about 1.10 M; and, more preferably, the concentration of the buffer is about 1.0 M; a typical volume of the buffer is from about 45 mL to about 55 mL; a preferable volume of the buffer is from about 47.5 mL to about 52.5 mL; and a more preferable volume of the buffer is about 50 mL.

Physiologically compatible buffers other than sodium bicarbonate, Tris buffer, and phosphate buffer can be used, as stated above. Still other physiologically compatible buffers can be used.

Typically, the sufficiently long period of time to enable the buffer to raise the pH in the bladder is about 15 minutes or more. More typically, the sufficiently long period of time to raise the pH in the bladder is from about 15 minutes to about 20 minutes. The sufficiently long period of time to raise the pH in the bladder can be adjusted according to patient tolerance and patient response.

The step of removing the physiologically compatible buffer of (1) from the bladder is typically performed by use of a catheter. Suitable catheters are well known to skilled practitioners and are commonly used in urology.

The composition administered in step (4) comprises: (i) a heparinoid; (ii) a local anesthetic; and (iii) a buffer. Typically, the composition administered in step (4) has a pH of from about 7.0 to about 7.4. In some alternatives as detailed below, additional components can be included in the composition administered in step (4).

The heparinoid is present in the composition in a quantity sufficient to treat the urinary tract disease or condition associated with bladder pain, such as, but not limited to, interstitial cystitis (also known as bladder pain syndrome (BPS) or bladder hypersensitivity syndrome (BHS)). The local anesthetic is also present in the composition in a quantity sufficient to treat the urinary tract disease or condition associated with bladder pain, such as, but not limited to, interstitial cystitis (also BPS or BHS). The buffer is present in the composition in a quantity such that from about 2% to about 45% of the local anesthetic is present in the composition in the free base (uncharged) form rather than the protonated (charged) form.

As used herein, “heparinoid” refers to any molecule comprising a glycosaminoglycan which refers to a molecule comprising a network of long, branched chains of sugars (e.g., heparin, chondroitin sulfate, heparan sulfate, hyaluronic acid, keratan sulfate, dermatan sulfate, hyaluronan, sodium pentosanpolysulfate, and the like) and optimally further comprising smaller, nitrogen-containing molecules (e.g. low molecular weight molecules). It is not meant to limit the present invention to any one glycosaminoglycan (GAG) or source of GAG. GAG molecules include but are not limited to low molecular weight (LMW) GAGs, naturally derived GAGs, biotechnologically prepared GAGs, chemically modified GAGs, synthetic GAGs, and the like. Heparinoids can also be comprised of pentoses instead of hexoses (GAGs are comprised of hexoses) such as pentosanpolysulfate. It is not meant to limit the present invention to any one heparinoid molecule or source of heparinoid molecule. As used herein, “heparin” refers to a heterogeneous group of straight-chain anionic glycosaminoglycans, as described above, with a molecular weight ranging from 2,000 to 40,000 Da. In some embodiments, heparin is a higher molecular weight species ranging from 8,000-40,000 daltons. As used herein, “low-molecular-weight heparins” refers to a lower molecular weight (LMVV) species ranging from 2,000 to 8,000 daltons. Sodium pentosanpolysulfate can range from 2,000 to 6,000 daltons. Also included within the scope of the invention are polymers such as dalteparin or enoxaparin. LMW heparins are made by enzymatic or chemical controlled hydrolysis of unfractionated heparin and have very similar chemical structure as unfractionated heparin except for some changes that may have been introduced due to the enzymatic or chemical treatment. While not intending to limit the mechanism of action of the invention's compositions, the mechanism of action of these drugs may be similar to that of full-length heparin. LMW heparins are usually isolated from bulk heparin. In one embodiment, heparin or another heparinoid is a heparin salt. As used herein, the phrases “pharmaceutically acceptable salts,” “a pharmaceutically acceptable salt thereof” or “pharmaceutically accepted complex” for the purposes of this invention are equivalent and refer to derivatives prepared from pharmaceutically acceptable non-toxic acids or bases including inorganic acids and bases and organic acids and bases.

Because of the negative charges of these polysaccharides due to the occurrence of sulfate groups and/or carboxylic acid groups in them, they are administered in the form of salts, with an appropriate cation to neutralize the negative charges on the acid groups. Typically, the cation is sodium. However, other physiologically tolerable counterions that do not induce urinary tract dysfunctions, such as magnesium, aluminum, calcium, ammonium, or salts made from physiologically acceptable organic bases such as, but not limited to, trimethylamine, triethylamine, morpholine, pyridine, piperidine, picoline, dicyclohexylamine, N,N′-dibenzylethylenediamine, 2-hydroxyethylamine, bis-(2-hydroxyethyl)amine, tri-(2-hydroxyethyl)amine, dibenzylpiperidine, N-benzyl-p-phenethylamine, dehydroabietylamine, N,N′-bisdehydroabietylamine, glucamine, N-methylglucamine, collidine, quinine, quinoline, and basic amino acids such as lysine and arginine, can be used. These cationic counterions can alternatively be used as the counterions with anionic buffers such as bicarbonate, as well. Sodium is typically employed as the positively-charged counterion as indicated above; accordingly, a preferred form of heparin is heparin sodium in which sodium acts as the counterion. These salts may be prepared by methods known to those skilled in the art. However, it is generally undesirable to use potassium as a counterion due to its role in the etiology of the conditions and syndromes being treated. Other polysaccharides that have the required activity include, but are not limited, to dextran sulfate and carrageenan. Other glycosaminoglycans can be used in methods according to the invention, including low molecular weight (LMW) glycosaminoglycans, naturally derived glycosaminoglycans, biotechnologically prepared glycosaminoglycans, chemically modified glycosaminoglycans, and synthetic glycosaminoglycans and linear anionic polysaccharides comprised of pentoses. Reference to a heparinoid that possesses a negative charge at physiological pH, such as heparin, without specific reference to a counterion, is to be understood as including all possible counterions that do not interfere with the physiological activity of the heparin or other components of the composition and do not create incompatibility with any other components of the composition.

In some embodiments, a heparinoid comprises a heparin-like molecule (e.g. heparan sulfate). For example, a heparin-like molecule such as heparan sulfate is a glycosaminoglycan with a structure similar to heparin with the difference being that heparan sulfate has undergone less polymerization than heparin and so has more glucuronic acid and N-acetyl glucosamine than heparin. Heparan sulfate contains fewer sulfate groups, so is somewhat less acidic. Heparin exists in a variety of forms characterized by different degrees of sulfation. Typically, heparin has a molecular weight of from about 2 kDa to about 40 kDa. Heparin and heparan sulfate are both characterized by repeating units of disaccharides containing a uronic acid (glucuronic or iduronic acid) and glucosamine, which is either N-sulfated or N-acetylated. The sugar residues may be further 0-sulfated at the C-6 and C-3 positions of the glucosamine and the C-2 position of the uronic acid. There are at least 32 potential unique disaccharide units in this class of compounds. Five examples of sugars occurring in heparin are: (1) α-L-iduronic acid 2-sulfate; (2) 2-deoxy-2-sulfamino-α-D-glucose 6-sulfate; (3) β-D-glucuronic acid; (4) 2-acetamido-2-deoxy-α-D-glucose; and (5) α-L-iduronic acid.

Typically, the heparinoid is selected from the group consisting of heparin, chondroitin sulfate, heparan sulfate, hyaluronic acid, keratan sulfate, dermatan sulfate, hyaluronan, sodium pentosanpolysulfate, dalteparin and enoxaparin. Particularly preferred heparinoids include heparin, heparan sulfate, chondroitin sulfate, hyaluronic acid, and sodium pentosanpolysulfate. A more particularly preferred heparinoid is heparin, such as heparin sodium. The heparin can be a heparin that has a molecular weight from about 2,000 daltons to about 8,000 daltons; alternatively, the heparin can be a heparin that has a molecular weight of from about 8,000 daltons to about 40,000 daltons.

A preferred form of heparin is heparin sodium, although, as described above, other counterions can be used. The quantity of heparin in compositions administered according to methods of the present invention in step (4) as described above can range from about 1000 units to about 250,000 units per unit dose of the composition; any intermediate quantity of heparin, such as, but not limited to, 1,000 units, 5,000 units, 10,000 units, 15,000 units, 20,000 units, 25,000 units, 30,000 units, 35,000 units, 40,000 units, 45,000 units, 50,000 units, 55,000 units, 60,000 units, 65,000 units, 70,000 units, 75,000 units, 80,000 units, 85,000 units, 90,000 units, 95,000 units, 100,000 units, 110,000 units, 120,000 units, 130,000 units, 140,000 units, 150,000 units, 160,000 units, 170,000 units, 180,000 units, 190,000 units, 200,000 units, 210,000 units, 220,000 units, 230,000 units, 240,000 units, or 250,000 units per unit dose of the composition can be used. As used herein, a “unit dose” refers to the dosage of heparin or other component of a composition administered according to methods of the present invention that is normally administered in a single treatment. As expressed in milligrams, these quantities of heparin range from about 0.5 mg to about 1250 mg per unit dose, including but not limited to 1 mg, 5 mg, 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, or 1250 mg. Suitable quantities of heparinoids other than heparin can be determined by one of ordinary skill in the art based on the molecular weight of the heparinoid to be used. Typically, the concentration of the heparin of the composition administered in methods according to the present invention from about 1,000 units of heparin per milliliter to about 6,000 units of heparin per milliliter of the composition. The concentration of the heparin of the composition administered in methods according to the present invention can be selected from the group consisting of 1,000 units, 1,500 units, 2,000 units, 2,500 units, 3,000 units, 3,500 units, 4,000 units, 4,500 units, 5,000 units, 5,500 units, and 6,000 units per milliliter of the composition.

The quantity of heparinoid in the composition can vary depending on the subject, the severity and course of the disease, the subject's health, the response to treatment, pharmacokinetic considerations such as liver and kidney function, and the judgment of the treating physician.

In accordance with the practice of the invention, merely by way of example, when the heparinoid is sodium pentosanpolysulfate, the quantity of heparinoid in the composition may be about 1 mg to about 600 mg of sodium pentosanpolysulfate per unit dose (for example about 100 mg to about 600 mg per unit dose of sodium pentosanpolysulfate). In accordance with the practice of the invention, merely by way of example, when the heparinoid is heparan sulfate, the amount of heparinoid in the composition may be about 0.5 mg to about 10,000 mg of heparan sulfate per unit dose (for example about 100 mg to about 300 mg per unit dose of heparan sulfate). In accordance with the practice of the invention, merely by way of example, when the heparinoid is hyaluronic acid, the amount of heparinoid in the composition may be about 5 mg to about 600 mg of hyaluronic acid per unit dose (for example about 10 mg to about 100 mg per unit dose of hyaluronic acid). In accordance with the practice of the invention, merely by way of example, when the heparinoid is chondroitin sulfate, the amount of heparinoid in the composition may be about 1 mg to about 10,000 mg of chondroitin sulfate per unit dose (for example about 100 mg to about 300 mg per unit dose of chondroitin sulfate). In accordance with the practice of the invention, merely by way of example, when the heparinoid is heparin sodium, the amount of heparinoid in the composition may be about 10 mg to about 1000 mg of heparin sodium per unit dose.

When the heparinoid is heparin, preferred quantities of heparin per unit dose of the composition include about 40,000 units, about 50,000 units, or about 60,000 units of heparin per unit dose of the composition.

Typically, the local anesthetic included in the composition of step (4) is a local anesthetic of the amide class that possesses a protonatable tertiary amino group that can form a positively charged quaternary amino group when protonated. Suitable local anesthetics include, but are not limited to, lidocaine, bupivacaine, etidocaine, mepivacaine, ropivacaine, dibucaine, dexivacaine, levobupivacaine, pyrrocaine, trimecaine, and rodocaine. Alternatively, other local anesthetics, including local anesthetics that are not local anesthetics of the amide class or local anesthetics that do not possess a protonatable tertiary amino group that can form a positively charged quaternary amino group when protonated can be used, including, but not limited to, benzocaine, tetracaine, pramoxine, procaine, chloroprocaine, dyclonine, oxybuprocaine, proparacaine, propoxycaine, diamocaine, hexylcaine, and risocaine. Suitable local anesthetics are typically sodium channel blockers.

Particularly preferred local anesthetics include, but are not limited to, lidocaine, bupivacaine, and mepivacaine. A more particularly preferred local anesthetic is lidocaine; preferably, the lidocaine is in the form of lidocaine hydrochloride, in which the chloride acts as a counterion. As used herein, the recitation of a local anesthetic includes all salts of that local anesthetic that are compatible with the desired pH, the buffer used, and any counterions present; the recitation of a local anesthetic is not intended to limit the salt form or counterion used beyond these criteria. Specifically, reference to an local anesthetic that possesses a positive charge at physiological or near-physiological pH, such as lidocaine, without specific reference to a counterion, is to be understood as including all possible counterions that do not interfere with the physiological activity of the lidocaine or other components of the composition and do not create incompatibility with any other components of the composition.

The quantity of local anesthetic in the composition of step (4) will vary depending on the subject, the severity and course of the disease, the subject's health, the response to treatment, pharmacokinetic considerations such as liver and kidney function, and the judgment of the treating physician. For example, when the local anesthetic is lidocaine, such as lidocaine hydrochloride, the amount of lidocaine in the composition may be in the range of about 10 mg to about 400 mg per unit dose, any intermediate quantity of lidocaine, such as 10 mg, 20 mg, 30 mg, 40 mg. 50 mg, 60 mg, 70 mg, 80 mg, 90 mg, 100 mg, 110 mg, 120 mg. 130 mg, 140 mg, 150 mg, 160 mg, 170 mg, 180 mg, 190 mg, 200 mg, 220 mg, 240 mg, 260 mg, 280 mg, 300 mg, 320 mg, 340 mg, 360 mg, 380 mg, or 400 mg per unit dose of the composition can be used. Typically, the concentration of the lidocaine of the composition is from about 5 mg/mL to about 20 mg/mL. For example, the amount of lidocaine can be 10 mL of 1% lidocaine per unit dose or 16 mL of 2% lidocaine per unit dose. In one preferred embodiment, the composition comprises 200 mg of lidocaine as lidocaine hydrochloride per unit dose. Suitable quantities of local anesthetics other than lidocaine can be determined by one of ordinary skill in the art based on the molecular weight and anesthetic potency of the local anesthetic to be used.

The buffer in the composition used in step (4) of a method according to the present invention can be, but is not limited to, phosphate buffer, bicarbonate buffer, Tris (Tris(hydroxymethyl)aminomethane) buffer, MOPS buffer (3-(N-morpholino)propanesulfonic acid), HEPES (N-(2-hydroxyethyl)piperazine-N-(2-ethanesulfonic acid) buffer, ACES (2-[(2-amino-2-oxoethyl)amino]ethanesulfonic acid) buffer, ADA (N-(2-acetamido)2-iminodiacetic acid) buffer, AMPSO (3-[(1,1-dimethyl-2-hydroxyethyl)amino]-2-propanesulfonic acid) buffer, BES (N,N-bis(2-hydroxyethyl)-2-aminoethanesulfonic acid buffer, Bicine (N,N-bis(2-hydroxyethylglycine) buffer, Bis-Tris (bis-(2-hydroxyethyl)imino-tris(hydroxymethyl)methane buffer, CAPS (3-(cyclohexylamino)-1-propanesulfonic acid) buffer, CAPSO (3-(cyclohexylamino)-2-hydroxy-1-propanesulfonic acid) buffer, CHES (2-(N-cyclohexylamino)ethanesulfonic acid) buffer, DIPSO (3-[N,N-bis(2-hydroxyethyl)amino]-2-hydroxy-propanesulfonic acid) buffer, HEPPS (N-(2-hydroxyethylpiperazine)-N′-(3-propanesulfonic acid) buffer, HEPPSO (N-(2-hydroxyethyl)piperazine-N′-(2-hydroxypropanesulfonic acid) buffer, MES (2-(N-morpholino)ethanesulfonic acid) buffer, triethanolamine buffer, imidazole buffer, glycine buffer, ethanolamine buffer, MOPSO (3-(N-morpholino)-2-hydroxypropanesulfonic acid) buffer, PIPES (piperazine-N,N′-bis(2-ethanesulfonic acid) buffer, POPSO (piperazine-N,N′-bis(2-hydroxypropaneulfonic acid) buffer, TAPS (N-tris[hydroxymethyl)methyl-3-aminopropanesulfonic acid) buffer; TAPSO (3-[N-tris(hydroxymethyl)methylamino]-2-hydroxy-propanesulfonic acid) buffer, TES (N-tris(hydroxymethyl)methyl-2-aminoethanesulfonic acid) buffer, tricine (N-tris(hydroxymethyl)methylglycine buffer), 2-amino-2-methyl-1,3-propanediol buffer, and 2-amino-2-methyl-1-propanol buffer, or a combination thereof. Particularly preferred buffers are bicarbonate buffer, phosphate buffer, Tris buffer or a combination thereof. When the buffer is bicarbonate buffer, the bicarbonate buffer is preferably sodium bicarbonate.

Because phosphate can bind up to three hydrogen ions, it can exist in several forms, including dihydrogen phosphate (H₂PO₄⁻), the monohydrogen phosphate (HPO₄²⁻), and the phosphate ion itself (PO₄³⁻). The pKₐ of the first ionization of phosphoric acid (H₃PO₄) to produce dihydrogen phosphate is about 2.12. The pKₐ of the ionization of dihydrogen phosphate to produce monohydrogen phosphate is about 7.21. The pKₐ of the ionization of monohydrogen phosphate to produce phosphate ion is about 12.67. The relative proportions of dihydrogen phosphate, monohydrogen phosphate, and phosphate ion present at a specified pH can readily be determined by use of the Henderson-Hasselbalch equation. Typically, when phosphate buffer is employed, it is employed as dihydrogen phosphate in view of the pH ranges involved; however, it is also possible to employ monohydrogen phosphate and add an alkalinizing agent such as sodium hydroxide to raise the pH to the desired value. Alternatively, a combination of monohydrogen phosphate and dihydrogen phosphate can be employed. Although it is possible to use other hydroxides such as potassium hydroxide, it is generally preferred to use sodium hydroxide in preference to potassium hydroxide in view of the potential role of potassium ion in the etiology of a number of lower urinary tract conditions. Phosphate buffer is a preferred buffer in some alternatives because it is more physiologically acceptable to the bladder and is normally present in urine.

In general, it is preferred to use an alkalinizing agent such as sodium hydroxide to achieve the final pH, rather than the buffer itself. The use of the alkalinizing agent to achieve the final pH results in greater stability of the acute-acting anesthetic, particularly lidocaine.

In one preferred alternative, the composition of step (4) is alkalinized lidocaine/heparin in phosphate buffer that contains 240 mg of lidocaine and 60,000 units of heparin in phosphate buffer with a pH of 7.2-7.3. In this preferred alternative, the volume of the alkalinized lidocaine/heparin in phosphate buffer that contains 240 mg of lidocaine and 60,000 units of heparin in phosphate buffer with a pH of 7.2-7.3 is about 20 mL per unit dose.

Other, optional, components, can be included in the composition; the composition can include one or more of these additional optional components. Such additional components can include:

(1) an osmolar component that provides an isotonic or nearly isotonic solution compatible with human cells and blood;

(2) a compound that enables persistence of the composition to the surface of the bladder epithelium in a quantity sufficient to treat, ameliorate, or prevent a lower urinary tract disorder;

(3) an antibacterial agent in a quantity sufficient to treat, ameliorate, or prevent a lower urinary tract disorder;

(4) an antifungal agent in a quantity sufficient to treat, ameliorate, or prevent a lower urinary tract disorder;

(5) a vasoconstrictor in a quantity sufficient to treat, ameliorate, or prevent a lower urinary tract disorder;

(6) a preservative; and

(7) an anti-inflammatory agent.

When present, the optional osmolar component is a salt, such as sodium chloride, or a sugar or a combination of two or more of these components. The sugar may be a monosaccharide such as dextrose, a disaccharide such as sucrose or lactose, a polysaccharide such as dextran 40, dextran 60, or starch, or a sugar alcohol such as mannitol. It should be obvious to those skilled in the art that all components of the solution contribute to the osmolarity of the solution but to achieve an isotonic or near-isotonic solution, the contributions of those components should be taken into account to ensure that the proper proportion of osmolar component is added and an excess of osmolar component is not added which would result in a hypertonic solution. In fact, when the composition as described above includes heparin sodium as the heparinoid, lidocaine hydrochloride as the anesthetic, and sodium bicarbonate as the buffer, the osmolar contributions of the sodium ion from the heparin sodium and sodium bicarbonate, the chloride ion from the lidocaine hydrochloride, and the carbonate/bicarbonate ion from the sodium bicarbonate are sufficient not to require an additional osmolar component. Similarly, when phosphate buffer is used, the osmolar contributions of the sodium ion and the phosphate ion are typically sufficient not to require an additional osmolar component. However, in some alternatives, an additional osmolar component can be used.

If an antibacterial agent is present, the antibacterial agent can be selected from the group consisting of a sulfonamide, a penicillin, a combination of trimethoprim plus sulfamethoxazole, a quinolone, methenamine, nitrofurantoin, a cephalosporin, a carbapenem, an aminoglycoside, a tetracycline, a macrolide, and gentamicin. Suitable sulfonamides include, but are not limited to, sulfanilamide, sulfadiazine, sulfamethoxazole, sulfisoxazole, sulfamethizole, sulfadoxine, and sulfacetamide. Suitable penicillins include, but are not limited to, methicillin, nafcillin, oxacillin, cloxacillin, dicloxacillin, ampicillin, amoxicillin, bacampicillin, carbenicillin, ticarcillin, mezlocillin, and piperacillin. Suitable quinolones include, but are not limited to, nalidixic acid, levofloxacin, cinoxacin, norfloxacin, ciprofloxacin, orfloxacin, sparfloxacin, lomefloxacin, fleroxacin, pefloxacin, and amifloxacin. Suitable cephalosporins include, but are not limited to, cephalothin, cephazolin, cephalexin, cefadroxil, cefamandole, cefoxatin, cefaclor, cefuroxime, loracarbef, cefonicid, cefotetan, ceforanide, cefotaxime, cefpodoxime proxetil, ceftizoxime, ceftriaxone, cefoperazone, ceftazidime, and cefepime. Suitable carbepenems include, but are not limited to, imipenem, meropenem, and aztreonam. Suitable aminoglycosides include, but are not limited to, netilmycin and gentamicin. Suitable tetracyclines include, but are not limited to, tetracycline, oxytetracycline, demeclocycline, minocycline, doxycycline, and chlortetracycline. Suitable macrolides include, but are not limited to, erythromycin, clarithromycin, and azithromycin.

If an antifungal agent is present, the antifungal agent can be selected from the group consisting of amphotericin B, itraconazole, ketoconazole, fluconazole, miconazole, and flucytosine.

If a vasoconstrictor is present, the vasoconstrictor can be epinephrine.

If a compound that enables persistence of the composition to the surface of the bladder epithelium is present, the compound is typically an activatable gelling agent. The activatable gelling agent is typically a thermoreversible gelling agent. The thermoreversible gelling agent can be selected from the group consisting of Pluronics F127 gel, Lutrol gel, N-isopropylacrylamide, ethylmethacrylate, N-acryloxysuccinimide, xyloglucan sols of 1-2%, graft copolymers of pluronic and poly(acrylic acid), pluronic-chitosan hydrogels, and a [poly(ethylene glycol)-poly[lactic acid-co-glycolic acid]-poly(ethylene glycol)] (PEG-PLGA-PEG) copolymer.

If a preservative is present, the preservative can be selected from the group consisting of parabens, chlorobutanol, phenol, sorbic acid, and thimerosal. However, typically, compositions that are administered in step (4) of the method according to the present invention do not require a preservative component and meet stability requirements without it. However, in some alternatives, it can be desirable to include a preservative component.

If an anti-inflammatory agent is present, the anti-inflammatory agent can be a steroid or a non-steroidal anti-inflammatory drug (NSAID). Suitable steroids and non-steroidal anti-inflammatory agents are known in the art. Suitable steroids include, but are not limited to, hydrocortisone, cortisone, beclomethasone dipropionate, betamethasone, dexamethasone, prednisone, methylprednisolone, triamcinolone, fluocinolone acetonide, and fludrocortisone. Suitable non-steroidal anti-inflammatory drugs include, but are not limited to, acetylsalicylic acid (aspirin), sodium salicylate, choline magnesium trisalicylate, salsalate, diflunisal, sulfasalazine, olsalazine, acetaminophen, indomethacin, sulindac, tolmetin, diclofenac, ketorolac, ibuprofen, naproxen, flurbiprofen, ketoprofen, fenoprofin, oxaprozin, mefenamic acid, meclofenamic acid, piroxicam, meloxicam, nabumetone, rofecoxib, celecoxib, etodolac, nimesulide, aceclofenac, alclofenac, alminoprofen, amfenac, ampiroxicam, apazone, araprofen, azapropazone, bendazac, benoxaprofen, benzydamine, bermoprofen, benzpiperylon, bromfenac, bucloxic acid, bumadizone, butibufen, carprofen, cimicoxib, cinmetacin, cinnoxicam, clidanac, clofezone, clonixin, clopirac, darbufelone, deracoxib, droxicam, eltenac, enfenamic acid, epirizole, esflurbiprofen, ethenzamide, etofenamate, etoricoxib, felbinac, fenbufen, fenclofenac, fenclozic acid, fenclozine, fendosal, fentiazac, feprazone, filenadol, flobufen, florifenine, flosulide, flubichin methanesulfonate, flufenamic acid, flufenisal, flunixin, flunoxaprofen, fluprofen, fluproquazone, furofenac, ibufenac, imrecoxib, indoprofen, isofezolac, isoxepac, isoxicam, licofelone, lobuprofen, lomoxicam, lonazolac, loxaprofen, lumaricoxib, mabuprofen, miroprofen, mofebutazone, mofezolac, morazone, nepafanac, niflumic acid, nitrofenac, nitroflurbiprofen, nitronaproxen, orpanoxin, oxaceprol, oxindanac, oxpinac, oxyphenbutazone, pamicogrel, parcetasal, parecoxib, parsalmide, pelubiprofen, pemedolac, phenylbutazone, pirazolac, pirprofen, pranoprofen, salicin, salicylamide, salicylsalicylic acid, satigrel, sudoxicam, suprofen, talmetacin, talniflumate, tazofelone, tebufelone, tenidap, tenoxicam, tepoxalin, tiaprofenic acid, tiaramide, tilmacoxib, tinoridine, tiopinac, tioxaprofen, tolfenamic acid, triflusal, tropesin, ursolic acid, valdecoxib, ximoprofen, zaltoprofen, zidometacin, and zomepirac.

If any of these optional components, i.e., the osmolar component, the compound that enables persistence of the composition to the surface of the bladder epithelium, the antibacterial component, the antifungal compound, the vasoconstrictor, the preservative, or the anti-inflammatory agent, are present, they are typically added after a stable solution including the heparinoid, the acute-acting anesthetic, and the buffer has been prepared. However, other alternatives for preparation of the composition of step (4) of a method according to the present invention when the composition includes one or more additional components are known in the art and can be used.

In some alternatives, the pharmaceutical composition to be administered in step (4) of the method according to the present invention can further include a suitable pharmaceutically acceptable carrier or excipient. These carriers and excipients include, but are not limited to: ion exchangers; alumina; aluminum stearate; lecithin, serum proteins, such as human serum albumin; glycine; sorbic acid; potassium sorbate; partial glyceride mixtures of saturated vegetable fatty acids; phosphate buffered saline solution; water; emulsions (e.g. oil/water emulsion); salts or electrolytes such as disodium hydrogen phosphate; sodium chloride; or zinc salts; colloidal silica; magnesium trisilicate; polyvinyl pyrrolidone; cellulose-based substances; and polyethylene glycol.

Methods for preparing compositions for administration in step (4) of the method according to the present invention are disclosed in PCT Patent Application Publication No. WO 2018/191412 by Vickery et al.

A particularly preferred composition for administration in step (4) of the method according to the present invention is alkalinized lidocaine/heparin in phosphate buffer that contains 240 mg of lidocaine and 60,000 units of heparin in phosphate buffer per unit dose with a pH of 7.2-7.3.

Typically, the volume of the composition administered in step (4) is from about 15 mL to about 25 mL. Preferably, the volume of the composition administered in step (4) is from about 17.5 mL to 22.5 mL. More preferably, the volume of the composition administered in step (4) is about 20 mL. However, other optimum volumes of the composition administered in step (4) can be determined for particular patients by a skilled practitioner, dependent on factors such as the size of the bladder, the particular disease or condition being treated, the severity of the disease or condition being treated, the tolerance of the patient to the instillation of large volumes of buffer into the bladder, other diseases or conditions affecting the urinary tract, including the bladder and ureters, and other medications being administered to the patient.

Typically, the composition administered in step (4) is left in the bladder for from about 40 minutes to about 50 minutes. Preferably, the composition administered in step (4) is left in the bladder for from about 42.5 minutes to about 47.5 minutes. More preferably, the composition administered in step (4) is left in the bladder for about 45 minutes. The time that the composition administered in step (4) is left in the bladder can be determined for particular patients by a skilled practitioner, dependent on factors such as the size of the bladder, the particular disease or condition being treated, the severity of the disease or condition being treated, the tolerance of the patient to the instillation of large volumes of buffer into the bladder, other diseases or conditions affecting the urinary tract, including the bladder and ureters, and other medications being administered to the patient.

In other alternatives, patients can be administered additional agents in therapeutically effective quantities to control the symptoms of interstitial cystitis or other diseases or conditions associated with bladder pain. These additional agents include, but are not limited to, oral non-steroidal anti-inflammatory drugs (NSAIDs) such as acetylsalicylic acid, acetaminophen, or ibuprofen; detroloxybutynin chloride; tolterodine; mesna; and dimethylsulfoxide.

Another alternative of the present invention is a multipart kit for the treatment of a disease or condition associated with bladder pain, comprising, separately packaged:

(1) one or more unit doses, separately packaged, of the physiologically compatible buffer administered in step (1) of the method according to the present invention;

(2) one or more unit doses of the composition, separately packaged, administered in step (4) of the method according to the present invention; and

(3) instructions for use of the kit.

The instructions for use of the kit can include, for example, specific diseases and conditions that the kit can be used to treat, including their symptoms and other diagnostic criteria, volumes of the solutions included in the kit to be administered, the durations for administration of the physiologically compatible buffer of (1) and the composition of (2), and other instructions to doctors, technicians, and other medical personnel.

Additionally, the kit may contain a catheter for each unit dose of the physiologically compatible buffer administered in step (1) of the method according to the present invention included in the kit and a catheter for each unit dose of the composition administered in step (4) of the method according to the present invention included in the kit.

Alternatives for the buffer included in component (1) of the kit and for the composition included in component (2) of the kit are as described above. For the composition included in component (2) of the kit, the alternatives for the heparinoid, the local anesthetic, the buffer, and, if present, the optional components, are as described above. Suitable volumes for the buffer of component (1) of the kit and for the composition of component (2) of the kit are also as described above.

### Advantages of the Invention

The present invention provides improved methods for the treatment of diseases and conditions associated with bladder pain, especially interstitial cystitis, as well as kits including components for use in the methods. The methods of the present invention are particularly well suited to treatment of interstitial disease patients who have not responded at all, or who have insufficiently responded, to previous treatments. The methods of the present invention are well tolerated by patients and can be used together with other methods for treating diseases or conditions associated with bladder pain.

Kits according to the present invention possess industrial applicability as articles of manufacture. Methods according to the present invention possess industrial applicability for the preparation of a medicament to treat lower urinary tract diseases and conditions.

With respect to ranges of values, the invention encompasses each intervening value between the upper and lower limits of the range to at least a tenth of the lower limit's unit, unless the context clearly indicates otherwise. Moreover, the invention encompasses any other stated intervening values and ranges including either or both of the upper and lower limits of the range, unless specifically excluded from the stated range.

Unless defined otherwise, the meanings of all technical and scientific terms used herein are those commonly understood by one of ordinary skill in the art to which this invention belongs. One of ordinary skill in the art will also appreciate that any methods and materials similar or equivalent to those described herein can also be used to practice or test this invention.

The publications and patents discussed herein are provided solely for their disclosure prior to the filing date of the present application. Nothing herein is to be construed as an admission that the present invention is not entitled to antedate such publication by virtue of prior invention. Further the dates of publication provided may be different from the actual publication dates which may need to be independently confirmed.

All the publications cited are incorporated herein by reference in their entireties, including all published patents, patent applications, and literature references, as well as those publications that have been incorporated in those published documents. However, to the extent that any publication incorporated herein by reference refers to information to be published, applicants do not admit that any such information published after the filing date of this application to be prior art.

As used in this specification and in the appended claims, the singular forms include the plural forms. For example the terms “a,” “an,” and “the” include plural references unless the content clearly dictates otherwise. Additionally, the term “at least” preceding a series of elements is to be understood as referring to every element in the series. The inventions illustratively described herein can suitably be practiced in the absence of any element or elements, limitation or limitations, not specifically disclosed herein. Thus, for example, the terms “comprising,” “including,” “containing,” etc. shall be read expansively and without limitation. As used herein, the transitional phrase “comprising” also encompasses the transitional phrases “consisting essentially of” and “consisting of” unless the transitional phrases with a narrower meaning are expressly excluded. Additionally, the terms and expressions employed herein have been used as terms of description and not of limitation, and there is no intention in the use of such terms and expressions of excluding any equivalents of the future shown and described or any portion thereof, and it is recognized that various modifications are possible within the scope of the invention claimed. Thus, it should be understood that although the present invention has been specifically disclosed by preferred embodiments and optional features, modification and variation of the inventions herein disclosed can be resorted by those skilled in the art, and that such modifications and variations are considered to be within the scope of the inventions disclosed herein. The inventions have been described broadly and generically herein. Each of the narrower species and subgeneric groupings falling within the scope of the generic disclosure also form part of these inventions. This includes the generic description of each invention with a proviso or negative limitation removing any subject matter from the genus, regardless of whether or not the excised materials specifically resided therein. In addition, where features or aspects of an invention are described in terms of the Markush group, those schooled in the art will recognize that the invention is also thereby described in terms of any individual member or subgroup of members of the Markush group. It is also to be understood that the above description is intended to be illustrative and not restrictive. Many embodiments will be apparent to those of in the art upon reviewing the above description. The scope of the invention should therefore, be determined not with reference to the above description, but should instead be determined with reference to the appended claims, along with the full scope of equivalents to which such claims are entitled. Those skilled in the art will recognize, or will be able to ascertain using no more than routine experimentation, many equivalents to the specific embodiments of the invention described. Such equivalents are intended to be encompassed by the following claims.

1. A method for treating a disease or condition associated with bladder pain comprising the steps of:
(a) instilling into the bladder of a subject with a disease or condition associated with bladder pain a quantity and concentration of a physiologically compatible buffer to raise the pH of the bladder epithelium and bladder interstitial tissue sufficiently to reduce acidosis associated with the disease or condition;
(b) allowing the physiologically compatible buffer of (a) to remain in the bladder for a period sufficiently long to enable the buffer to raise the pH in the bladder tissues;
(c) removing the physiologically compatible buffer of (a) from the bladder to prevent the precipitation of anesthetic by the high volume and concentration of physiologically compatible buffer of (a); and
(d) instilling into the bladder a quantity of a composition comprising: (i) a heparinoid; (ii) a local anesthetic; and (iii) a buffer, wherein the composition has a pH of from about 7.0 to about 7.4 to treat the disease or condition associated with bladder pain.

2. The method of claim 1 wherein the disease or condition associated with bladder pain is selected from the group consisting of bacterial cystitis, fungal/yeast cystitis, vulvar vestibulitis, vulvodynia, dyspareunia, urethral syndrome, and endometriosis in women; prostatitis and chronic pelvic pain syndrome in men; and radiation-induced cystitis, chemotherapy-induced cystitis, interstitial cystitis, and overactive bladder in men or women.

3. The method of claim 2 wherein the disease or condition associated with bladder pain is interstitial cystitis.

4. The method of claim 1 wherein the physiologically compatible buffer of step (a) is selected from the group consisting of phosphate buffer, bicarbonate buffer, Tris (Tris(hydroxymethyl)aminomethane) buffer, MOPS buffer (3-(N-morpholino)propanesulfonic acid), HEPES (N-(2-hydroxyethyl)piperazine-N-(2-ethanesulfonic acid) buffer, ACES (2-[(2-amino-2-oxoethyl)amino]ethanesulfonic acid) buffer, ADA (N-(2-acetamido)2-iminodiacetic acid) buffer, AMPSO (3-[(1,1-dimethyl-2-hydroxyethyl)amino]-2-propanesulfonic acid) buffer, BES (N,N-bis(2-hydroxyethyl)-2-aminoethanesulfonic acid buffer, Bicine (N,N-bis(2-hydroxyethylglycine) buffer, Bis-Tris (bis-(2-hydroxyethyl)imino-tris(hydroxymethyl)methane buffer, CAPS (3-(cyclohexylamino)-1-propanesulfonic acid) buffer, CAPSO (3-(cyclohexylamino)-2-hydroxy-1-propanesulfonic acid) buffer, CHES (2-(N-cyclohexylamino)ethanesulfonic acid) buffer, DIPSO (3-[N,N-bis(2-hydroxyethyl)amino]-2-hydroxy-propanesulfonic acid) buffer, HEPPS (N-(2-hydroxyethylpiperazine)-N′-(3-propanesulfonic acid) buffer, HEPPSO (N-(2-hydroxyethyl)piperazine-N′-(2-hydroxypropanesulfonic acid) buffer, IVIES (2-(N-morpholino)ethanesulfonic acid) buffer, triethanolamine buffer, imidazole buffer, glycine buffer, ethanolamine buffer, MOPSO (3-(N-morpholino)-2-hydroxypropanesulfonic acid) buffer, PIPES (piperazine-N,N′-bis(2-ethanesulfonic acid) buffer, POPSO (piperazine-N,N′-bis(2-hydroxypropaneulfonic acid) buffer, TAPS (N-tris[hydroxymethyl)methyl-3-aminopropanesulfonic acid) buffer; TAPSO (34N-tris(hydroxymethyl)methylamino]-2-hydroxy-propanesulfonic acid) buffer, TES (N-tris(hydroxymethyl)methyl-2-aminoethanesulfonic acid) buffer, tricine (N-tris(hydroxymethyl)methylglycine buffer), 2-amino-2-methyl-1,3-propanediol buffer, and 2-amino-2-methyl-1-propanol buffer, and a combination thereof.

5. The method of claim 4 wherein the physiologically compatible buffer is selected from the group consisting of phosphate buffer, Tris buffer, and bicarbonate buffer.

6.-8. (canceled)

9. The method of claim 5 wherein the buffer is bicarbonate buffer, wherein the bicarbonate buffer is sodium bicarbonate and wherein the volume of sodium bicarbonate buffer is from about 40 mL to about 55 mL or less dependent on the maximum volume tolerated by the patient.

10.-15. (canceled)

16. The method of claim 5 wherein the volume of Tris buffer or phosphate buffer is from about 45 mL to about 55 mL.

17.-19. (canceled)

20. The method of claim 1 wherein the sufficiently long period of time to enable the buffer to raise the pH in the bladder wall is about 15 minutes or more.

21. (canceled)

22. The method of claim 1 wherein the composition administered in step (d) includes the heparinoid in a quantity sufficient to treat the urinary tract disease or condition associated with bladder pain and includes the local anesthetic in a quantity sufficient to treat the urinary tract disease or condition associated with bladder pain.

23.-24. (canceled)

25. The method of claim 1 wherein the heparinoid of the composition of step (d) is selected from the group consisting of heparin, chondroitin sulfate, heparan sulfate, hyaluronic acid, keratan sulfate, dermatan sulfate, sodium pentosanpolysulfate, dalteparin and enoxaparin.

26. The method of claim 25 wherein the heparinoid is selected from the group consisting of heparin, heparan sulfate, chondroitin sulfate, hyaluronic acid, and sodium pentosanpolysulfate.

27. The method of claim 26 wherein the heparinoid is heparin or heparin sodium.

28.-31. (canceled)

32. The method of claim 26 wherein the heparinoid is sodium pentosanpolysulfate.

33.-39. (canceled)

40. The method of claim 27 wherein the heparinoid is heparin and wherein the quantity of heparin per unit dose of the composition of step (d) is about 40,000 units, about 50,000 units, or about 60,000 units of heparin per unit dose of the composition.

41. The method of claim 1 wherein the local anesthetic included in the composition of step (d) is a local anesthetic of the amide class that possesses a protonatable tertiary amino group that can form a positively charged quaternary amino group when protonated.

42. The method of claim 41 wherein the local anesthetic is selected from the group consisting of lidocaine, bupivacaine, etidocaine, mepivacaine, ropivacaine, dibucaine, dexivacaine, levobupivacaine, pyrrocaine, trimecaine, and rodocaine.

43. (canceled)

44. The method of claim 43 wherein the local anesthetic is lidocaine or lidocaine hydrochloride.

45. The method of claim 44 wherein the local anesthetic is lidocaine and wherein the quantity of lidocaine in the composition of step (d) is from about 10 mg to about 400 mg per unit dose of the composition.

46.-48. (canceled)

49. The method of claim 44 wherein the local anesthetic is lidocaine and wherein the composition of step (d) includes 200 mg of lidocaine hydrochloride per unit dose of the composition.

50. The method of claim 1 wherein the buffer of the composition of step (d) is selected from the group consisting of phosphate buffer, bicarbonate buffer, Tris (Tris(hydroxymethyl)aminomethane) buffer, MOPS buffer (3-(N-morpholino)propanesulfonic acid), HEPES (N-(2-hydroxyethyl)piperazine-N-(2-ethanesulfonic acid) buffer, ACES (2-[(2-amino-2-oxoethyl)amino]ethanesulfonic acid) buffer, ADA (N-(2-acetamido)2-iminodiacetic acid) buffer, AMPSO (3-[(1,1-dimethyl-2-hydroxyethyl)amino]-2-propanesulfonic acid) buffer, BES (N,N-bis(2-hydroxyethyl)-2-aminoethanesulfonic acid buffer, Bicine (N,N-bis(2-hydroxyethylglycine) buffer, Bis-Tris (bis-(2-hydroxyethyl)imino-tris(hydroxymethyl)methane buffer, CAPS (3-(cyclohexylamino)-1-propanesulfonic acid) buffer, CAPSO (3-(cyclohexylamino)-2-hydroxy-1-propanesulfonic acid) buffer, CHES (2-(N-cyclohexylamino)ethanesulfonic acid) buffer, DIPSO (3-[N,N-bis(2-hydroxyethyl)amino]-2-hydroxy-propanesulfonic acid) buffer, HEPPS (N-(2-hydroxyethylpiperazine)-N′-(3-propanesulfonic acid) buffer, HEPPSO (N-(2-hydroxyethyl)piperazine-N′-(2-hydroxypropanesulfonic acid) buffer, IVIES (2-(N-morpholino)ethanesulfonic acid) buffer, triethanolamine buffer, imidazole buffer, glycine buffer, ethanolamine buffer, MOPSO (3-(N-morpholino)-2-hydroxypropanesulfonic acid) buffer, PIPES (piperazine-N,N′-bis(2-ethanesulfonic acid) buffer, POPSO (piperazine-N,N′-bis(2-hydroxypropaneulfonic acid) buffer, TAPS (N-tris[hydroxymethyl)methyl-3-aminopropanesulfonic acid) buffer; TAPSO (34N-tris(hydroxymethyl)methylamino]-2-hydroxy-propanesulfonic acid) buffer, TES (N-tris(hydroxymethyl)methyl-2-aminoethanesulfonic acid) buffer, tricine (N-tris(hydroxymethyl)methylglycine buffer), 2-amino-2-methyl-1,3-propanediol buffer, and 2-amino-2-methyl-1-propanol buffer, or a combination thereof.

51. The method of claim 1 wherein the composition of step (d) is alkalinized lidocaine/heparin in phosphate buffer that contains 240 mg of lidocaine and 60,000 units of heparin in phosphate buffer with a pH of 7.2-7.3.

52. (canceled)

53. The method of claim 1 wherein the composition of step (d) includes at least one additional component.

54. The method of claim 53 wherein the additional component is:
(a) an osmolar component that provides an isotonic or nearly isotonic solution compatible with human cells and blood;
(b) an antibacterial agent;
(c) an antifungal agent;
(d) a vasoconstrictor;
(e) a compound that enables persistence of the composition to the surface of the bladder epithelium and in the interstitial tissue of the bladder wall;
(f) a preservative; or
(g) an anti-inflammatory agent.

55.-68. (canceled)

69. The method of claim 1 wherein the volume of the composition administered in step (d) is from about 15 mL to about 25 mL.

70.-71. (canceled)

72. The method of claim 1 wherein the composition administered in step (d) is left in the bladder for from about 40 minutes to about 50 minutes or less depending on patient tolerability.

73. The method of claim 1 further comprising the step of administering a therapeutically effective quantity of an additional agent to control the symptoms of interstitial cystitis or other diseases or conditions associated with bladder pain wherein the additional agent is selected from the group consisting of an oral non-steroidal anti-inflammatory drug, detroloxybutynin chloride, tolterodine, mesna, and dimethyl sulfoxide.

74. (canceled)

75. A multipart kit for the treatment of a disease or condition associated with bladder pain comprising:
(a) one or more unit doses, separately packaged, of a physiologically compatible buffer to raise the pH of the bladder epithelium sufficiently to reduce acidosis associated with the disease and condition;
(b) one or more unit doses, separately packaged, of a composition comprising: (i) a heparinoid; (ii) a local anesthetic; and (iii) a buffer, wherein the composition has a pH of from about 7.0 to about 7.4 to treat the disease or condition associated with bladder pain; and
(c) instructions for use of the kit.

76. The multipart kit of claim 75 wherein the multipart kit further comprises a catheter for each unit dose of the physiologically compatible buffer of (a) and a catheter for each unit dose of the composition of (b).

77.-100. (canceled)

101. The multipart kit of claim 75 wherein the composition of (b) includes at least one additional component, wherein the at least one additional component is selected from the group consisting of:
(a) an osmolar component that provides an isotonic or nearly isotonic solution compatible with human cells and blood;
(b) an antibacterial agent;
(c) an antifungal agent;
(d) a vasoconstrictor;
(e) a compound that enables persistence of the composition to the surface of the bladder epithelium;
(f) a preservative; and
(g) an anti-inflammatory agent.

102.-107. (canceled)
